(19) United States
(12) Patent Application Publication
(10) Pub. No.: US 2024/0245832 A1
(43) Pub. Date: Jul. 25, 2024

(54) BIOENGINEERED ENDOTHELIAL CONSTRUCTS

(71) Applicants: THE UNIVERSITY OF SYDNEY, Sydney, NSW (AU); SOUTH EASTERN SYDNEY LOCAL HEALTH DISTRICT, Caringbah, NSW (AU)

(72) Inventors: Jingjing YOU, Ryde, NSW (AU); Gerard SUTTON, Chatswood, NSW (AU); Morgan OVERMASS, North Willoughby (AU); Constantinos PETSOGLOU, Sydney, NSW (AU); Sheng HUA, Sydney, NSW (AU); Li WEN, Sydney, NSW (AU)

(21) Appl. No.: 18/551,085

(22) PCT Filed: Mar. 18, 2022
(86) PCT No.: PCT/AU2022/050248

§ 371 Date: Sep. 18, 2023

(30) Foreign Application Priority Data
Mar. 18, 2021 (AU) 2021900795

(51) Int. Cl. A61L 27/38; A61L 27/24; A61L 27/36; A61L 27/54; C12N 5/079

(52) U.S. Cl. CPC A61L 27/3808; A61L 27/24; A61L 27/3616; A61L 27/3687; A61L 27/54; C12N 5/0621; A61L 2300/414; A61L 2430/16; C12N 2500/32; C12N 2500/38; C12N 2501/165; C12N 2533/54; C12N 2533/56; C12N 2537/10

(57) ABSTRACT

The present invention relates to bioengineered collagen constructs and methods for the preparation and use of said constructs. The constructs and methods may find application in, but are not limited to, the provision of replacement tissue and/or cells, and/or the delivery of agents to biological targets such as tissues and cells. The constructs and methods may also find application in, but are not limited to, replacing endothelial tissue.

### INCORPORATION BY CROSS-REFERENCE

This application claims priority from Australian provisional patent application number 2021900795, filed on 18 Mar. 2021, the entire contents of which are incorporated herein by cross-reference.

### TECHNICAL FIELD

The present invention relates generally to the fields of biology and medicine. More specifically, the present invention relates to bioengineered collagen constructs and methods for the preparation and use of said constructs. The constructs and methods may find application in the provision of replacement tissue and/or cells, and/or the delivery of agents to biological targets such as tissues and cells.

### BACKGROUND

When disease or injury results in significant damage to biological tissue, clinicians are often restricted by the availability of suitable donor tissue for use in transplantation. Bioengineered constructs provide an exciting solution to this problem, but are restricted by requirements for strength and flexibility, in addition to the need for the synthetic material to be compatible with biological tissue and and/or cells.

One example of an area with a shortage of donor tissue available for transplants is corneal disease and trauma. Currently, traditional eye banks represent a storage and distribution centre for donated tissue. Ideally, these eye banks would have the capacity to also source and culture corneal tissue and to bioengineer additional tissue for therapeutic applications.

The cornea is the clear, protective outer layer of the eye. It allows light to pass through the pupil and is the primary refractive element of the eye's optical system. It serves as a barrier against bacteria, dirt and other harmful substances. The cornea consists of five layers: the outer epithelium, Bowman's layer, the stroma, Descemet's membrane, and the inner endothelium. Descemet's membrane is the basement membrane for the corneal endothelium and is a dense, thick, relatively transparent and cell-free matrix that separates the posterior corneal stroma from the underlying endothelium. The corneal stroma accounts for approximately 90% of the overall thickness of the cornea and is mostly made up of collagen.

Corneal blindness is the second major cause of blindness worldwide. Causes of corneal blindness include diseases such as trachoma, onchocerciasis, leprosy, ophthalmia neonatorum, and xerophthalmia, and other processes such as ocular trauma, corneal ulceration, and complications arising from the use of traditional eye medicines.

Corneal injuries represent the most common ophthalmic emergency presentation in Australia and approximately 75% of all cases are due to the presence of foreign bodies or abrasions in the cornea. These injuries alone are estimated to cost the Australian population more than $155 million per year and, if not treated effectively, can lead to infection and scarring, resulting in permanently impaired vision.

In mild cases, a damaged cornea is able to regenerate via normal healing pathways. In other cases, however, the cornea's normal healing mechanism is insufficient, leading to the formation of non-healing defects which can result in corneal melting, corneal neovascularisation, loss of transparency, infection, scarring and diminished vision to the point of blindness.

Corneal endothelial disease is one example of a condition affecting the cornea which would benefit from improved constructs for tissue replacement. Whilst the surgical treatment of corneal endothelial disease has evolved in recent years through the use of lamellar transplants, this surgery remains technically difficult and utilises precious donor corneal tissue. Recognized complications include subluxation of the donor tissue.

Current medical treatments for corneal injuries include antibiotics, eye pads, sutures and surgical glues, which may help with minor issues. However, they do not adequately address issues arising in more advanced situations including pain relief, infection and/or the development of scar tissue. Infection represents a significant complication and often requires hospitalisation. Scarring, which is common in severe corneal injuries, can lead to permanent vision loss. In such cases, corneal transplantation is the only option for visual rehabilitation, but a shortage of donor corneas exists worldwide.

Many of the aforementioned issues are not restricted to corneal injuries and disease, and also prevail in the case of damage and/or deterioration of other body tissues.

A need exists for improved constructs and methods for the provision of replacement tissue and/or cells, and/or the delivery of agents to biological targets such as tissues and cells.

### SUMMARY OF THE INVENTION

The present invention alleviates at least one of the problems associated with current supply and methods for the replacement of biological tissue and/or the delivery of agents to biological targets such as tissues and cells. The present inventors have developed constructs comprising type IV collagen which are transparent, crosslinkable, strong, flexible and/or printable. Surprisingly, the constructs may provide a unique way of culturing endothelial cells, for example, human corneal endothelial cells.

Without limitation, the constructs and methods described herein are generally useful for the delivery of agents (e.g. cells, drugs and/or or other substances) to biological targets (e.g. tissue, membranes, cells) and may find application, for example, in endothelial tissue replacement, including human corneal endothelial tissue.

The present invention relates at least in part to the following embodiments:

Embodiment 1. A construct comprising a composition, wherein the composition comprises:
type IV collagen;
one or more crosslinking agents;
endothelial cells; and
platelet lysate and/or a component thereof.

Embodiment 2. A construct comprising a composition, wherein the composition comprises:
type IV collagen;
one or more crosslinking agents; and
endothelial cells.

Embodiment 3. The construct of embodiment 1 or embodiment 2, wherein the endothelial cells are corneal endothelial cells.

Embodiment 4. The construct of any one of embodiments 1 to 3, wherein the endothelial cells are human endothelial cells.

Embodiment 5. The construct of any one of embodiments 1, 3 or 4, wherein the platelet lysate is human platelet lysate.

Embodiment 6. The construct of any one of embodiments 1 or 3 to 5, wherein the component of platelet lysate is any one or more of: fibrinogen, basic fibroblast growth factor (bFGF), transforming growth factor (TGF-β), insulin-like growth factor (IGF-1), brain-derived neurotrophic factor (BDNF), vascular endothelial growth factor (VEGF), epidermal growth factor (EGF), hepatocyte growth factor (HGF), platelet-derived growth factor (PDGF), IgG, albumin.

Embodiment 7. The construct of any one of embodiments 1 or 3 to 6, wherein the component of platelet lysate is fibrinogen.

Embodiment 8. The construct of embodiment 7, wherein the fibrinogen is human fibrinogen.

Embodiment 9. The construct of any one of embodiments 1 to 8, wherein the construct and/or the composition further comprises sodium ions and/or calcium ions.

Embodiment 10. The construct of any one of embodiments 1 to 9, wherein the one or more crosslinking agents are capable of activation by UV light, blue light, green light or white light.

Embodiment 11. The construct of any one of embodiments 1 to 10, wherein the one or more crosslinking agents comprise riboflavin.

Embodiment 12. The construct of any one of embodiments 1 to 11, wherein the construct comprises at least one layer comprising the type IV collagen and the one or more crosslinking agents, and wherein each layer is produced by crosslinking a solution comprising:
6-24 mg/ml type IV collagen, and
0.04-0.15 M sodium ions and/or 0.008-0.4 M calcium ions.

Embodiment 13. The construct of embodiment 12, wherein the solution comprises:
(i) 6-24 mg/ml type IV collagen, 0.06-0.1 M sodium ions, and 0.01-0.04 M calcium ions; or
(ii) 3-15 mg/ml type IV collagen, 0.06-0.08 M sodium ions, and 0.015-0.03 M calcium ions; or
(iii) 4-12 mg/ml type IV collagen, 0.06-0.07 M sodium ions, and 0.018-0.02 M calcium ions.

Embodiment 14. The construct of embodiment 12 or embodiment 13, wherein the solution comprises:
(i) less than 24 mg/ml type IV collagen;
(ii) more than 0.04 M sodium ions; and
(iii) more than 0.008 M calcium ions.

Embodiment 15. The construct of any one of embodiments 12 to 14, wherein the solution comprises 0.01-0.1 mg riboflavin.

Embodiment 16. The construct of any one of embodiments 1 to 15, wherein the construct comprises two or more layers comprising the type IV collagen and the one or more crosslinking agents, and wherein each of the two or more layers has been crosslinked to at least one other layer.

Embodiment 17. The construct of any one of embodiments 12 to 16, wherein the construct comprises at least one additional layer comprising type I collagen and one or more crosslinking agents, wherein the additional layer is produced by crosslinking a solution comprising:
3-15 mg/ml type I collagen;
0.135-0.5 M sodium ions and/or 0.008-0.4 M calcium ions.

Embodiment 18. The construct of embodiment 17, wherein each layer has been crosslinked to at least one other layer.

Embodiment 19. The construct of any one of embodiments 1 to 18, wherein the construct comprises a first layer comprising the collagen and the one or more crosslinking agents that has been individually crosslinked prior to crosslinking to one or more other layers.

Embodiment 20. The construct of any one of embodiments 1 to 11, wherein the construct comprises at least one layer comprising type IV collagen, type I collagen and the one or more crosslinking agents, and wherein each layer is produced by crosslinking a solution comprising:
6-24 mg/ml type IV collagen, and
3-15 mg/ml type I collagen.

Embodiment 21. The construct of embodiment 20, wherein the solution comprises 0.01-0.1 mg riboflavin.

Embodiment 22. The construct of embodiment 20 or embodiment 21, wherein the construct comprises two or more layers comprising the type IV collagen, type I collagen and the one or more crosslinking agents, and wherein each of the two or more layers has been crosslinked to at least one other layer.

Embodiment 23. The construct of any one of embodiments 20 to 22, wherein the construct comprises a first layer comprising the collagen and the one or more crosslinking agents that has been individually crosslinked prior to crosslinking to one or more other layers.

Embodiment 24. The construct of any one of embodiments 1 to 23, wherein the construct and/or the composition further comprises mammalian cells.

Embodiment 25. The construct of embodiment 24, wherein the mammalian cells comprise or consist of human cells.

Embodiment 26. The construct of any one of embodiments 1 to 25, wherein the construct and/or the composition further comprises any one or more of: a culture medium, growth factors, hormones, matrix proteins, glycoproteins, vitamins, ions, ion sources, fibronectin, amino acids, antibiotics, anaesthetics, factor XIII, Fetal Bovine Serum (FBS), Fetal Calf Serum (FCS), human serum, platelet lysate, human platelet lysate, therapeutic drugs.

Embodiment 27. The construct of embodiment 26, wherein the construct and/or the composition comprises a culture medium comprising the ions and amino acids.

Embodiment 28. The construct of embodiment 26 or embodiment 27, wherein:
(i) the growth factors comprise VEGF and/or FGF; and/or
(ii) the vitamins comprise riboflavin; and/or
(iii) the matrix proteins comprise type I collagen; and/or
(iv) the matrix proteins comprise laminin.

Embodiment 29. The construct of any one of embodiments 1 to 28, wherein the construct and/or the composition comprises ions, and wherein the ions are components of an ionic salt included in the construct and/or the composition.

Embodiment 30. The construct of any one of embodiments 1 to 29, wherein the type IV collagen is neutralised.

Embodiment 31. The construct of any one of embodiments 1 to 30, wherein the construct and/or the composition further comprises any one or more of: type VIII collagen, laminin, nidogen, perlecan.

Embodiment 32. A method of preparing a construct, the method comprising:
(i) providing a composition, wherein the composition is produced by crosslinking a solution comprising:
   type IV collagen, and
   one or more crosslinking agents; and
(ii) adding:
   endothelial cells, and
   platelet lysate and/or a component thereof.

Embodiment 33. A method of preparing a construct, the method comprising:
(i) providing a composition, wherein the composition is produced by crosslinking a solution comprising:
   type IV collagen, and
   one or more crosslinking agents; and
(ii) adding endothelial cells.

Embodiment 34. A method of culturing endothelial cells, the method comprising:
(i) providing a composition, wherein the composition is produced by crosslinking a solution comprising:
   type IV collagen, and
   one or more crosslinking agents; and
(ii) adding:
   endothelial cells, and
   platelet lysate and/or a component thereof.

Embodiment 35. A method of culturing endothelial cells, the method comprising:
(i) providing a composition, wherein the composition is produced by crosslinking a solution comprising:
   type IV collagen, and
   one or more crosslinking agents; and
(ii) adding endothelial cells.

Embodiment 36. The method of any one of embodiments 32 to 35, wherein the composition is produced by crosslinking a second solution comprising:
type I collagen, and
one or more crosslinking agents,
prior to (ii).

Embodiment 37. The method of any one of embodiments 32 to 35, wherein the solution further comprises type I collagen.

Embodiment 38. The method of any one of embodiments 32 to 37, wherein the endothelial cells are corneal endothelial cells.

Embodiment 39. The method of any one of embodiments 32, 34 or 36 to 38, wherein the platelet lysate is human platelet lysate.

Embodiment 40. The method of any one of embodiments 32, 34 or 36 to 39, wherein the component of platelet lysate is any one or more of: fibrinogen, bFGF, TGF-β, IGF-1, BDNF, VEGF, EGF, HGF, PDGF, IgG, albumin.

Embodiment 41. The method of any one of embodiments 32, 34 or 36 to 40, wherein the component of platelet lysate is fibrinogen.

Embodiment 42. The method of embodiment 41, wherein the fibrinogen is human fibrinogen.

Embodiment 43. The method of any one of embodiments 32 to 42, further comprising adding sodium ions and/or calcium ions in step (i) and/or step (ii).

Embodiment 44. The method of any one of embodiments 32 to 43, wherein the one or more crosslinking agents are capable of activation by UV light, blue light, green light or white light.

Embodiment 45. The method of any one of embodiments 32 to 44, wherein the one or more crosslinking agents comprise riboflavin.

Embodiment 46. The method of any one of embodiments 32 to 45, wherein the composition comprises at least one layer comprising the crosslinked solution, and wherein the solution comprises:
6-24 mg/ml type IV collagen, and
0.04-0.15 M sodium ions and/or 0.008-0.4 M calcium ions.

Embodiment 47. The method of embodiment 46, wherein the solution comprises:
(i) 6-24 mg/ml type IV collagen, 0.06-0.1 M sodium ions, and 0.01-0.04 M calcium ions; or
(ii) 3-15 mg/ml type IV collagen, 0.06-0.08 M sodium ions, and 0.015-0.03 M calcium ions; or
(iii) 4-12 mg/ml type IV collagen, 0.06-0.07 M sodium ions, and 0.018-0.02 M calcium ions.

Embodiment 48. The method of embodiment 46 or embodiment 47, wherein the solution comprises:
(i) less than 24 mg/ml type IV collagen;
(ii) more than 0.04 M sodium ions; and
(iii) more than 0.008 M calcium ions.

Embodiment 49. The method of any one of embodiments 45 to 48, wherein the solution comprises 0.01-0.1 mg riboflavin.

Embodiment 50. The method of any one of embodiments 32 to 49, wherein the composition comprises two or more layers comprising the collagen and the one or more crosslinking agents, and wherein each of the two or more layers has been crosslinked to at least one other layer.

Embodiment 51. The method of any one of embodiments 32 to 50, wherein the composition comprises a first layer comprising the collagen and the one or more crosslinking agents that has been individually crosslinked prior to crosslinking to one or more other layers.

Embodiment 52. The method of embodiment 50 or embodiment 51, wherein at least one layer of the composition is crosslinked on a hydrophobic material.

Embodiment 53. The method of embodiment 52, wherein the hydrophobic material is silicone or parafilm.

Embodiment 54. The method of embodiment 50 or embodiment 51, wherein at least one layer of the composition is crosslinked on a cold material.

Embodiment 55. The method of embodiment 54, wherein the cold material is cold metal.

Embodiment 56. The method of any one of embodiments 32 to 55, further comprising adding mammalian cells in step (i) and/or step (ii).

Embodiment 57. The method of embodiment 56, wherein the mammalian cells comprise or consist of human cells.

Embodiment 58. The method of any one of embodiments 32 to 57, further comprising adding in step (i) and/or step (ii) any one or more of: a culture medium, growth factors, hormones, matrix proteins, glycoproteins, vitamins, ions, ion sources, fibronectin, amino acids, antibiotics, anaesthetics, factor XIII, FBS, FCS, human serum, platelet lysate, human platelet lysate, therapeutic drugs.

Embodiment 59. The method of embodiment 58, wherein the ions and/or amino acids are provided in a culture medium.

Embodiment 60. The method of embodiment 58 or embodiment 59, wherein:
(i) the growth factors comprise VEGF and/or FGF; and/or
(ii) the vitamins comprise riboflavin; and/or
(iii) the matrix proteins comprise type I collagen; and/or
(iv) the matrix proteins comprise laminin.

Embodiment 61. The method of any one of embodiments 32 to 60, further comprising adding ions as components of an ionic salt in step (i) and/or step (ii).

Embodiment 62. The method of any one of embodiments 32 to 61, wherein the type IV collagen is neutralised.

Embodiment 63. The method of any one of embodiments 32 to 62, further comprising adding in step (i) and/or step (ii) any one or more of: type VIII collagen, laminin, nidogen, perlecan.

Embodiment 64. A construct obtained or obtainable by the method of any one of embodiments 32, 33 or 36 to 63.

Embodiment 65. A method of replacing endothelial tissue, the method comprising applying the construct of any one of embodiments 1 to 31 or embodiment 64.

Embodiment 66. The method of embodiment 65, wherein the endothelial tissue comprises or consists of the corneal endothelium.

Embodiment 67. The method of embodiment 65 or embodiment 66, wherein the endothelial tissue is human endothelial tissue.

Embodiment 68. The method of any one of embodiments 65 to 67, wherein the endothelial tissue comprises or consists of Descemet's membrane.

Embodiment 69. A method of treating corneal injury or disease, the method comprising applying the construct of any one of embodiments 1 to 31 or embodiment 64.

Embodiment 70. A method of delivering agents to tissue, the method comprising applying the composition of any one of embodiments 1 to 31 or embodiment 64 to the tissue.

Embodiment 71. A construct of any one of embodiments 1 to 31 or embodiment 64 for use in replacing endothelial tissue.

Embodiment 72. The use of embodiment 71, wherein the endothelial tissue comprises or consists of the corneal endothelium.

Embodiment 73. The use of embodiment 71 or embodiment 72, wherein the endothelial tissue is human endothelial tissue.

Embodiment 74. The use of any one of embodiments 71 to 73, wherein the tissue comprises or consists of Descemet's membrane.

Embodiment 75. A construct of any one of embodiments 1 to 31 or embodiment 64 for use in treating corneal injury or disease.

Embodiment 76. A construct of any one of embodiments 1 to 31 or embodiment 64 for use in delivering agents to tissue.

Embodiment 77. A kit, package or device for producing a construct comprising a composition, wherein the composition comprises:
type IV collagen, and
one or more crosslinking agents;
and wherein the kit, package or device further comprises:
endothelial cells, and
platelet lysate and/or a component thereof.

Embodiment 78. A kit, package or device for producing a construct comprising a composition, wherein the composition comprises:
type IV collagen, and
one or more crosslinking agents;
and wherein the kit, package or device further comprises endothelial cells.

Embodiment 79. The kit, package or device of embodiment 77 or embodiment 78, wherein the composition further comprises type I collagen.

Embodiment 80. The kit, package or device of embodiment 77 or embodiment 78, wherein the kit, package or device is for producing a further composition, wherein the composition comprises:
type IV collagen,
type I collagen, and
one or more crosslinking agents.

Embodiment 81. Use of a kit, package or device for preparing a construct comprising a composition, wherein the composition comprises:
type IV collagen, and
one or more crosslinking agents;
and wherein the kit package or device further comprises:
endothelial cells, and
platelet lysate and/or a component thereof.

Embodiment 82. Use of a kit, package or device for preparing a construct comprising a composition, wherein the composition comprises:
type IV collagen, and
one or more crosslinking agents;
and wherein the kit package or device further comprises:
endothelial cells.

Embodiment 83. The use of embodiment 81 or embodiment 82, wherein the composition further comprises type I collagen.

Embodiment 84. The use of embodiment 81 or embodiment 82, wherein the use is for producing a further composition, wherein the composition comprises:
type IV collagen,
type I collagen, and
one or more crosslinking agents.

Embodiment 85. The kit, package or device of any one of embodiments 77 to 80 or the use of any one of embodiments 80 to 84, wherein the endothelial cells are corneal endothelial cells.

Embodiment 86. The kit, package or device of any one of embodiments 77 to 80 or 85 or the use of any one of embodiments 80 to 85, wherein the endothelial cells are human endothelial cells.

Embodiment 87. The kit, package or device of any one of embodiments 77, 79, 80, 85 or 86 or the use of any one of embodiments 80, 81 or 83 to 86, wherein the platelet lysate is human platelet lysate.

Embodiment 88. The kit, package or device of any one of embodiments 77, 79, 80 or 85 to 87 or the use of any one of embodiments 80, 81 or 83 to 87, wherein the component of platelet lysate is any one or more of: fibrinogen, bFGF, TGF-β, IGF-1, BDNF, VEGF, EGF, HGF, PDGF, IgG, albumin.

Embodiment 89. The kit, package or device of any one of embodiments 77, 79, 80 or 85 to 88 or the use of any one of embodiments 80, 81 or 83 to 88, wherein the component of platelet lysate is fibrinogen.

Embodiment 90. The kit, package or device or the use of embodiment 89, wherein the fibrinogen is human fibrinogen.

Embodiment 91. The kit, package or device of any one of embodiments 77 to 80 or 85 to 90 or the use of any one of embodiments 80 to 90, wherein the construct and/or the composition further comprises sodium ions and/or calcium ions.

Embodiment 92. The kit, package or device of any one of embodiments 77 to 80 or 85 to 91 or the use of any one of embodiments 80 to 91, wherein the one or more crosslinking agents are capable of activation by UV light, blue light, green light or white light.

Embodiment 93. The kit, package or device of any one of embodiments 77 to 80 or 85 to 92 or the use of any one of embodiments 80 to 92, wherein the one or more crosslinking agents comprise riboflavin.

Embodiment 94. The kit, package or device of any one of embodiments 77 to 80 or 85 to 93 or the use of any one of embodiments 80 to 93, wherein the construct comprises at least one layer comprising the type IV collagen and the one or more crosslinking agents, and wherein each layer is produced by crosslinking a solution comprising:
6-24 mg/ml type IV collagen, and
0.04-0.15 M sodium ions and/or 0.008-0.4 M calcium ions.

Embodiment 95. The kit, package or device or the use of embodiment 94, wherein the solution comprises:
(i) 6-24 mg/ml type IV collagen, 0.06-0.1 M sodium ions, and 0.01-0.04 M calcium ions; or
(ii) 3-15 mg/ml type IV collagen, 0.06-0.08 M sodium ions, and 0.015-0.03 M calcium ions; or
(iii) 4-12 mg/ml type IV collagen, 0.06-0.07 M sodium ions, and 0.018-0.02 M calcium ions.

Embodiment 96. The kit, package or device or the use of embodiment 94 or embodiment 95, wherein the solution comprises:
(i) less than 24 mg/ml type IV collagen;
(ii) more than 0.04 M sodium ions; and
(iii) more than 0.008 M calcium ions.

Embodiment 97. The kit, package or device or the use of any one of embodiments 94 to 96, wherein the solution comprises 0.01-0.1 mg riboflavin.

Embodiment 98. The kit, package or device of any one of embodiments 77 to 80 or 85 to 97 or the use of any one of embodiments 80 to 97, wherein the construct comprises two or more layers comprising the type IV collagen and the one or more crosslinking agents, and wherein each of the two or more layers has been crosslinked to at least one other layer.

Embodiment 99. The kit, package or device of any one of embodiments 77 to 80 or 85 to 98 or the use of any one of embodiments 80 to 98, wherein the construct comprises a first layer comprising the type IV collagen and the one or more crosslinking agents that has been individually crosslinked prior to crosslinking to one or more other layers.

Embodiment 100. The kit, package or device of any one of embodiments 77 to 80 or 85 to 99 or the use of any one of embodiments 80 to 99, wherein the construct and/or the composition further comprises mammalian cells.

Embodiment 101. The kit, package or device or the use of embodiment 100, wherein the mammalian cells comprise or consist of human cells.

Embodiment 102. The kit, package or device of any one of embodiments 77 to 80 or 85 to 101 or the use of any one of embodiments 80 to 101, wherein the construct and/or the composition further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, FBS, FCS, human serum, platelet lysate, human platelet lysate, therapeutic drugs.

### Definitions

As used in this application, the singular form “a”, “an” and “the” include plural references unless the context clearly dictates otherwise. For example, the term “component” also includes a plurality of components.

As used herein, the term “comprising” means “including”. Variations of the word “comprising”, such as “comprise” and “comprises,” have correspondingly varied meanings. Thus, for example, a composition “comprising” component ‘A’ may consist exclusively of component ‘A’ or may include one or more additional components (e.g. component ‘B’ and/or component ‘C’).

As used herein, the term “subject” includes any animal of economic, social or research importance including bovine, equine, ovine, primate, avian and rodent species. Hence, a “subject” may be a mammal such as, for example, a human, or a non-human mammal.

As used herein, the term “tissue” will be understood to encompass both cells that are component/s of the tissue and organ/s formed from the tissue.

As used herein, the term “kit” refers to any delivery system for delivering materials. Such delivery systems include systems that allow for the storage, transport, or delivery of reaction reagents (e.g., labels, reference samples, supporting material, etc. in appropriate containers) and/or supporting materials (e.g., buffers, written instructions for performing an assay etc.) from one location to another. For example, kits may include one or more enclosures, such as boxes, containing the relevant reaction reagents and/or supporting materials.

As used herein, the term “about” when used in reference to a recited numerical value includes the recited numerical value and numerical values within plus or minus ten percent of the recited value.

As used herein, the term “plurality” means more than one. In certain specific aspects or embodiments, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any numerical value derivable therein, and any range derivable therein.

As used herein, the term “between” when used in reference to a range of numerical values encompasses the numerical values at each endpoint of the range. For example, calcium ions with a concentration of between 0.008 M and 0.4 M is inclusive of calcium ions with a concentration 0.008 M and calcium ions with a concentration 0.4 M.

As used herein, the term “more than” when used in reference to a numerical value will be understood to mean “greater than or equal to”. For example, more than 0.018 M calcium ions encompasses a concentration of 0.018 M calcium ions and all concentrations of calcium ions greater than 0.018 M.

As used herein, the term “less than” when used in reference to a numerical value will be understood to mean “less than or equal to”. For example, less than 15 mg/ml type IV collagen encompasses a concentration of 15 mg/ml type IV collagen and all concentrations of type IV collagen less than 15 mg/ml.

As used herein, the term “neutralised” when used to describe type IV collagen will be understood to mean that the pH of the collagen solution is between 6.7 and 7.6. For example, “neutralised” type IV collagen could have a pH between 6.8 and 7.5, or between 6.9 and 7.4, or between 7.0 and 7.3, or between 6.9 and 7.2, etc.

As used herein, the term “crosslink”, when used in reference to collagen, will be understood to mean an increase in intra- and intermolecular covalent bonds. Variations of the word “crosslink”, such as “crosslinked” and “crosslinking,” have correspondingly varied meanings. In some instances, collagen “crosslinking” will be understood to mean an increase in intra- and interfibrillar covalent bonds in collagen fibrils.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### BRIEF DESCRIPTION OF THE FIGURES

Preferred embodiments of the present invention will now be described by way of example only, with reference to the accompanying figures wherein:

FIG. 1 provides representative images of cell line corneal endothelial cells growing on a double and triple layer of col-4 respectively (FIG. 1A and FIG. 1B). When exposed to 5% hPL both the double-layered and triple layered col-4 membranes began to peel from the coverslip in the well by Day 3. FIG. 1C provides a representative image of the cell borders of corneal endothelial cells on a double layer of col-4 which remained adhered following mechanical disruption. FIG. 1A and FIG. 1B were taken at 10× magnification. FIG. 1C was taken at 20× magnification.

FIGS. 2A and 2B provide representative images of double layer col-4 membranes in culture at day 5. In FIG. 2A the membrane has been cultured in 5% hPL media for 5 days and the col-4 corneal endothelial sheet has now detached from the coverslip within the well. In FIG. 2B the membrane has been cultured in 5% FCS media for 5 days and whilst confluent shows no sign of detaching as a sheet. FIG. 2C provides a representative image of a detached sheet removed from the well, which is transparent and flat.

FIG. 3 provides representative images of cell recovery following mechanical disruption. A comparison is shown between recovery when col-4 remained for cells to potentially expand onto and when the col-4 film was torn. All images were taken at 10× magnification.

FIG. 4 provides representative images of col-4 membranes which have been marked with either a simple dot (FIG. 4A), which does not fade following washes in PBS, or with more complex shapes like an ‘F’ (FIG. 4B), which is an asymmetrical shape often used to mark Descemet's membranes in surgery to allow for orientation with the eye.

FIG. 5 provides representative images demonstrating the capacity of the col-4 membranes (in particular the double layered membranes as shown) to be aspirated into a Stryker injector (FIG. 5A) and to be stained with VisionBlue (FIG. 5B). The dashed lines in FIG. 5B represent the area remaining following trephining of the membrane by an 8.5 mm trephine.

FIG. 6 provides a representative image of a double layered col-4 membrane with corneal endothelial cells (shown in the image as the speckled surface present within the cornea) adhered to the stromal surface within a human cornea following 1 minute when minimal liquid is present.

FIG. 7 provides representative images of the assembly of the moulding platform. A: the supporting material—silicone. B: the intermediate layer—polyester film with a hole. C: the top layer—polyester film. D: assembled platform. E: the supporting material can also be paraffin. F: cold metal plate was the supporting material with intermediate layer on top. The bioink will be loaded to the intermediate layer and covered by the top layer.

FIG. 8 provides images of immunostaining of laminin (red) on collagen membranes. A: Col-4 membrane with laminin. B: Col-4 only membrane. The line in the top right corner of each image shows the border of membrane.

FIG. 9 provides phase contrast images of the four endothelial constructs. All showed high cell compatibility with primary corneal endothelial cells reaching confluence. A: Col-4 at 10.8 mg/mL. B: Col-1 and col-4 mixed. C: Col-1 layer with col-4 layer on the top.

FIG. 10 provides an image of a cell carrying collagen membrane which has self-detached from the culture dish.

FIG. 11 provides images taken following immunostaining. Corneal primary cells cultured on the representative membranes showed high expression of Na/K-ATPase compared to the control. A: Col-4 membrane at 10.8 mg/mL, B: Col-1 layer with col-4 layer on the top C: Control.

FIG. 12 provides images of membranes made by three supporting materials. A: Silicone, B: Paraffin and C: cold metal plate.

### DETAILED DESCRIPTION

The present inventors have developed constructs comprising type IV collagen which are transparent, crosslinkable, strong, flexible and/or printable. The constructs of the present invention may provide a unique way of culturing endothelial cells, for example, corneal endothelial cells and may provide a means of delivering agents to biological targets (e.g. organs, tissues, cells). The constructs may be used as a carrier for cells, for example, endothelial cells. The constructs described herein may be capable of mimicking Descemet's membrane in the cornea. The constructs may provide a replacement membrane and/or may allow endothelial healing. While suitable for application to the cornea, the constructs provide a platform for numerous applications in the areas of endothelial replacement and/or repair, and the delivery of agents by virtue of providing, for example, structural support, transparency, flexibility, viable cells, and other factors.

The constructs described herein may utilise biomaterial that mimics in vivo tissue and acts as a scaffold for cells to populate, and/or, through the manipulation of conditions, encourage the cells themselves to regenerate their surrounding matrix. In the context of their suitability for application to eye tissue the present inventors have, for example, addressed the difficulties of creating a construct that can embody the structural integrity of the tissue under treatment (e.g. Descemet's membrane in the cornea) whilst maintaining transparency and still being porous and biocompatible enough to allow for the infiltration, migration and/or proliferation of corneal endothelial cells and growth factors.

### Constructs

The present invention provides constructs suitable for endothelial tissue replacement, for example, the replacement of corneal endothelial tissue. The constructs may also be used for the delivery of agents to biological targets such as tissues and cells.

The constructs may comprise a composition comprising type IV collagen and one or more crosslinking agents. The constructs of the invention may comprise a composition comprising type IV collagen, type I collagen and one or more crosslinking agents. In some embodiments, the constructs further comprise endothelial cells. The constructs may comprise platelet lysate and/or a component thereof. The platelet lysate may be human platelet lysate. Non-limiting examples of components of platelet lysate include fibrinogen, basic fibroblast growth factor (bFGF), transforming growth factor (TGF-β), insulin-like growth factor (IGF-1), brain-derived neurotrophic factor (BDNF), vascular endothelial growth factor (VEGF), epidermal growth factor (EGF), hepatocyte growth factor (HGF), platelet-derived growth factor (PDGF), IgG and albumin. In some embodiments, the component of platelet lysate may be fibrinogen, which may be human fibrinogen. The construct may comprise one or more layers of a composition. The composition may comprise type IV collagen. The collagen used may be unmodified when compared to its naturally occurring counterpart. Modified type IV collagen may also be used. In some embodiments, the modifications are at the end of the collagen fibre.

The construct may comprise one or more layers of a composition comprising type IV collagen. Some constructs of the invention comprise one or more layers of a composition comprising type IV collagen and type I collagen. Additionally or alternatively, the construct may comprise one or more layers of a composition comprising type IV collagen crosslinked to one or more layers of a composition comprising type I collagen. The layers of the composition comprising type IV collagen and the composition comprising type I collagen may be alternating. In some embodiments of the invention, the construct comprises two layers, with one layer comprising a composition comprising type I collagen and the other layer comprising a composition comprising type IV collagen.

The composition used to produce the constructs of the present invention may further optionally comprise ions and/or one or more sources of ions. Non-limiting examples of suitable ions include calcium ions and sodium ions. Non-limiting examples of suitable ion sources include compounds comprising calcium (e.g. calcium chloride) and sodium (e.g. sodium chloride). Calcium ions and sodium ions may be present in the compositions together or individually. Ions may be present in the constructs and/or the composition.

The constructs and/or compositions may comprise endothelial cells, for example, corneal endothelial cells. The endothelial cells may be human endothelial cells. In some embodiments, the constructs and/or the compositions further comprise Fetal Calf Serum (FCS) and/or platelet lysate. The platelet lysate may be human platelet lysate. In further embodiments, the constructs and/or the compositions comprise fibrinogen, which may be human fibrinogen. The constructs and/or the compositions may also comprise laminin.

The present inventors have identified optimal relative concentrations of type IV collagen, type I collagen, sodium ions and/or calcium ions for the compositions which are used to produce the constructs of the present invention, some of which are described in the Examples and claims of the present application. It will be understood that the relative concentrations of type IV collagen, type I collagen, sodium ions and/or calcium ions disclosed are exemplary only.

The composition may comprise 6-24 mg/ml type IV collagen, 0.04-0.15 M sodium ions and/or 0.008-0.4 M calcium ions. The composition may comprise 1-20 mg/ml type IV collagen, 0.07-0.5 M sodium ions and/or 0.008-0.4 M calcium ions. In some embodiments, the composition may comprise 1-20 mg/ml type IV collagen, 0.06-0.25 M sodium ions and/or 0.008-0.1 M calcium ions. The composition may comprise 3-15 mg/ml type IV collagen, 0.06-0.25 M sodium ions and/or 0.008-0.4 M calcium ions. Other potential ranges for the components of the composition include 3-15 mg/ml type IV collagen, 0.06-0.1 M sodium ions and/or 0.01-0.05 M calcium ions. In some embodiments, the composition may comprise 4-12 mg/ml type IV collagen, 0.06-0.08 M sodium ions and/or 0.015-0.03 M calcium ions. Alternatively, the composition may comprise 5-10 mg/ml type IV collagen, 0.06-0.07 M sodium ions and/or 0.018-0.02 M calcium ions. In some embodiments, the composition may comprise less than 15 mg/ml type IV collagen and more than 0.06 M sodium ions and/or more than 0.018 M calcium ions.

The compositions of the present invention may further comprise one or more crosslinking agents. One non-limiting example of a suitable crosslinking agent is riboflavin. The riboflavin could be present at a concentration of 0.01-0.5% (w/v). The riboflavin could be present at an amount of about 0.01-0.1 mg. In some embodiments, the amount of riboflavin present is 0.01, 0.1 mg or any amount between these values. Light, such as UV light or blue light could be used to activate the riboflavin, crosslinking the composition. The person skilled in the art will be aware of other suitable crosslinking agents and/or light sources, for example, Rose Bengal dye and green light, both of which have been approved for several applications to the cornea. In some embodiments, Rose Bengal is used as a photocrosslinking agent and is activated by green light. Alternatively, Rose Bengal is used as a photocrosslinking agent and is activated by white light. In some embodiments, 0.01-0.5% (w/v) Rose Bengal is used for photocrosslinking. In further embodiments, compositions provided by crosslinking the collagen composition with Rose Bengal and a suitable light source will be coloured. In some embodiments, the colour will be pink. These coloured compositions may be used for monitoring collagen metabolism in tissue, or for other uses requiring tracking of collagen activity. The constructs of the present invention may be provided in their crosslinked form.

The constructs may comprise a single layer of a collagen composition which may have been crosslinked or may comprise two or more layers of the collagen composition. Each layer may have been crosslinked to the existing construct prior to the addition of the next layer. This may include the first layer of the construct, which may have been individually crosslinked prior to the addition of any other layers.

The constructs may be produced using a moulding platform. A suitable moulding platform may comprise a supporting surface, an intermediate material and a top material. The compositions may be crosslinked on a supporting surface made of hydrophobic materials. Non-limiting examples of suitable hydrophobic materials include silicone and parafilm. Crosslinking may also be carried out on a cold surface. The cold surface could be a metal surface. In some embodiments of the invention, an intermediate material may be used which may have a hollow centre. The hollow centre of the intermediate material may be used to transfer the composition onto the supporting surface. One non-limiting example of a suitable material for the intermediate layer is polyester. A top material may be added prior to compression with weight. One non-limiting example of a suitable material for the top layer is polyester.

The construct and/or the composition may further comprise cells. The cells may, for example, be mammalian cells (e.g. human cells, canine cells, feline cells, bovine cells, porcine cells, equine cells, caprine cells, hircine cells, murine cells, leporine cells, cricetine cells, musteline cells, or any combination thereof). The type of cells utilised will generally depend on the specific purpose for which the composition is to be used. For example, the cells may be of the same type as a tissue to which the composition is to be administered. Non-limiting examples of suitable cell types include eye cells including those of the central and/or peripheral corneal epithelium, bulbar and/or tarsal conjunctival epithelia, tarsal conjunctival stroma, corneal endothelium and/or lid margin; skin cells including but not limited to keratinocytes, melanocytes, Merkel cells, and Langerhans cells; and neural tissue cells including but not limited to neurons and glial cells. Other examples include epithelial cells, keratocytes, neuronal cells, and endothelial cells. The endothelial cells may be primary endothelial cells. In some embodiments, the cells may be hematopoietic stem cells, bone marrow stem cells, neural stem cells, epithelial stem cells, skin stem cells, muscle stem cells, adipose stem cells, pluripotent stem cells, induced pluripotent stem cells, embryonic stem cells, mesenchymal stem cells, or any combination thereof. In some embodiments, the cells may be neuronal cells. One non-limiting example of suitable epithelial cells is lens epithelial cells. A non-limiting example of suitable endothelial cells is corneal endothelial cells. The cells of the constructs and/or compositions may be autologous (i.e. self-derived from a given subject intended to receive the composition, or allogeneic (i.e. donor-derived).

The constructs and/or compositions of the present invention may comprise essential and/or non-essential amino acids. Non-limiting examples of suitable essential amino acids include isoleucine, leucine, lysine, methionine, phenylalanine, threonine, tryptophan, valine, cysteine, tyrosine, histidine and arginine.

The constructs and/or compositions of the present invention may comprise additional components (e.g. agent/s) including, but not limited to, fibronectin, anaesthetics, antibiotics, hormones (e.g. insulin), growth factors (e.g. human epidermal growth factor (hEGF), platelet derived growth factor, vascular endothelial growth factor, fibroblast growth factor (FGF), epithelial growth factor, transforming growth factor [including beta], and connective tissue growth factor), fibrin stabilizing factors (e.g. factor XIII), matrix protein/s (e.g. collagen [such as type I collagen], laminin, integrin), vitamins (e.g. vitamin C, riboflavin), glycoproteins (e.g. transferrin), Fetal Bovine Serum (FBS), Fetal Calf Serum (FCS), human serum, platelet lysate, human platelet lysate, therapeutic drugs and any combination thereof. In some embodiments, the composition and/or construct comprises a culture medium comprising the ions and amino acids. Non-limiting examples of suitable growth factors include vascular endothelial growth factor (VEGF) and fibroblast growth factor (FGF). The vitamins may be ascorbate (vitamin C, riboflavin). The matrix proteins could include, but are not limited to, type I collagen; and/or laminin.

The constructs and/or compositions of the present invention may include other suitable ingredients including water and/or culture medium (e.g. DMEM, DMEM/F-12, MEM, CnT-PR). The culture medium may comprise, for example, any one or more of Glycine, L-Alanine, L-Arginine hydrochloride, L-Asparagine-H2O, L-Aspartic acid, L-Cysteine hydrochloride-H2O, L-Cystine 2HCl, L-Glutamic Acid, L-Glutamine, L-Histidine hydrochloride-H2O, L-Isoleucine, L-Leucine, L-Lysine hydrochloride, L-Methionine, L-Phenylalanine, L-Proline, L-Serine, L-Threonine, L-Tryptophan, L-Tyrosine disodium salt dihydrate, L-Valine, Vitamins, Biotin, Choline chloride, D-Calcium pantothenate, Folic Acid, Niacinamide Pyridoxine hydrochloride, Riboflavin, Thiamine hydrochloride, Vitamin B12, i-Inositol, Inorganic Salts, Calcium Chloride (CaCl₂)) (anhyd.), Cupric sulfate (CuSO4-5H2O), Ferric Nitrate (Fe(NO3)3″9H2O), Ferric sulfate (FeSO4-7H2O), Magnesium Chloride (anhydrous), Magnesium Sulfate (MgSO4) (anhyd.), Potassium Chloride (KCl), Sodium Bicarbonate (NaHCO₃), Sodium Chloride (NaCl), Sodium Phosphate dibasic (Na2HPO4) anhydrous, Sodium Phosphate monobasic (NaH2PO4-, Zinc sulfate (ZnSO4-7H2O), Other Components, D-Glucose (Dextrose), Hypoxanthine Na, Linoleic Acid, Lipoic Acid, Putrescine 2HCl, Sodium Pyruvate, Thymidine, or any combination thereof. In some embodiments, ions are provided as components of an ionic salt included in the composition.

Non-limiting properties of the constructs/compositions include one or more of the following:
Non-Newtonian shear-thinning fluid properties, whereby the viscosity of the construct/composition may decrease as the shear-rate increases. In some embodiments, the viscosity of the constructs/compositions may be in the range of 0.01 and 1000 Pa·s at room temperature.
Optical clarity without impeding or without substantially impeding vision arising from transmittance of light, for example, over 90% in the visual colour range of 400-700 nm.
Suitability for 2D and/or 3D printing (e.g. bioprinting/extrusion printing) with capacity to maintain or substantially maintain shape/structure following printing.
Suitability for printing while maintaining the viability of cells within the composition/construct during the printing process.
Capacity to be provided in two- or three-dimensional structure with or without the inclusion of viable cells.
Capacity to sustain and/or promote the growth of cells (e.g. sustain and/or promote the expansion and/or growth of primary human cells such as epithelial cells (e.g. lens epithelial cells), keratocytes, neuronal cells, and endothelial cells (e.g. corneal endothelial cells).
Capacity to promote the formation of spheroid organoids.
Capacity for degradation by cells over time (e.g. 2-7 days).
Maintenance of cell viability over time (e.g. 7 days at 34° C.).
Capacity to adhere to various surfaces, including tissues, organs, membranes (e.g. mammalian and human tissues, organs, membranes).

### Preparation of Constructs

The constructs may be prepared by forming one or more layers of a composition. The construct may have been crosslinked. The composition in the one or more layers may be prepared by combining a plurality of different preparations. In some embodiments, lyophilised bovine type IV collagen may be used in the preparation of the compositions. Additionally or alternatively, human collagen may be used. The type IV collagen may be neutralised prior to the addition of ions and other components of the compositions. A person skilled in the art would recognise that various buffer solutions could be used to keep the collagen at physiological pH and to maintain solubility. In some embodiments of the invention, type IV collagen may be mixed in the preparation of the compositions.

In some embodiments of the invention, a single layer of the composition is applied to a surface to form the construct. The single layer may have been individually crosslinked. In further embodiments, one or more additional layers are added to a first layer. Each layer may be crosslinked to the existing construct prior to the addition of the next layer. No particular limitation exists as to the number of layers of composition which may form a construct. The layers may comprise, type IV collagen, type I collagen or a mixture of the two. Alternating layers of compositions comprising only type IV collagen and only type I collagen may be used to form a construct. In some embodiments, the construct has two layers; one layer comprises a composition comprising only type IV collagen and the other layer comprises a composition comprising only type I collagen.

The composition used to create the construct may comprise 6-24 mg/ml type IV collagen, 0.04-0.15 M sodium ions and/or 0.008-0.4 M calcium ions, or 1-20 mg/ml type IV collagen, 0.07-0.5 M sodium ions and/or 0.008-0.4 M calcium ions. In some embodiments, the composition may comprise 1-20 mg/ml type IV collagen, 0.06-0.25 M sodium ions and/or 0.008-0.1 M calcium ions. The composition may comprise 3-15 mg/ml type IV collagen, 0.06-0.25 M sodium ions and/or 0.008-0.4 M calcium ions. Other potential ranges for the components of the composition include 3-15 mg/ml type IV collagen, 0.06-0.1 M sodium ions and/or 0.01-0.05 M calcium ions. In some embodiments, the composition may comprise 4-12 mg/ml type IV collagen, 0.06-0.08 M sodium ions and/or 0.015-0.03 M calcium ions. Alternatively, the composition may comprise 5-10 mg/ml type IV collagen, 0.06-0.07 M sodium ions and/or 0.018-0.02 M calcium ions. In some embodiments, the composition may comprise less than 15 mg/ml type IV collagen and more than 0.06 M sodium ions and/or more than 0.018 M calcium ions.

The compositions used to create the constructs of the present invention may further comprise one or more crosslinking agents. One non-limiting example of a suitable crosslinking agent is riboflavin. The riboflavin could be present at an amount of about 0.1 mg. An amount of 0.01-0.1 mg/ml riboflavin could be present. The riboflavin could be present at a concentration of 0.01-0.5% (w/v). Light, such as UV light or blue light could be used to activate the riboflavin, crosslinking the composition. The person skilled in the art will be aware of other suitable crosslinking agents and/or light sources, for example, Rose Bengal dye and green light, both of which have been approved for several applications to the cornea. In some embodiments, Rose Bengal is used as a photocrosslinking agent and is activated by green light. Alternatively, Rose Bengal is used as a photocrosslinking agent and is activated by white light. In some embodiments, 0.01-0.5% (w/v) Rose Bengal is used for photocrosslinking. The constructs and/or compositions may comprise endothelial cells, for example, corneal endothelial cells. The endothelial cells may be human endothelial cells. In some embodiments, the constructs and/or the compositions further comprise Fetal Calf Serum (FCS) and/or platelet lysate.

In further embodiments, the constructs and/or the compositions further comprise fibrinogen, which may be human fibrinogen. The human fibrinogen may be added to the constructs at a concentration of 0.7-2 mg/ml. In some embodiments, the platelet lysate and/or the fibrinogen are added to the constructs for use as a culturing medium to culture the endothelial cells. Additionally or alternatively, a component of platelet lysate may be added. The platelet lysate may be human platelet lysate. Non-limiting examples of suitable components of platelet lysate include fibrinogen, basic fibroblast growth factor (bFGF), transforming growth factor (TGF-β), insulin-like growth factor (IGF-1), brain-derived neurotrophic factor (BDNF), vascular endothelial growth factor (VEGF), epidermal growth factor (EGF), hepatocyte growth factor (HGF), platelet-derived growth factor (PDGF), IgG and albumin.

In some embodiments, the invention provides devices and/or kits for the preparation of the constructs. The devices and/or kits may facilitate the separation of different preparations needed to form the constructs and/or compositions of the invention until use.

The devices and kits may further comprise a component providing a means to facilitate mixing of the two compartmentalised preparations such as, for example, by removal of barrier/s separating the first and second compartments, and/or by puncturing a seal or wall of either or both compartments. The skilled person will readily understand that various arrangements can be made for this purpose.

Additionally or alternatively, devices and kits may be configured in a manner that ensures mixing of the two compartmentalised preparations during or following release of the preparations from the device or kit.

In some embodiments, the devices and kits may comprise additional compartments comprising additional components to be used in generating the composition and/or construct (e.g., platelet lysate, ions, amino acids, cells, antibiotics, growth factors, fibrinogen stabilizing factors, anaesthetics and so on). The device or kit may be configured in such a way to facilitate mixing of these additional components with each other and/or with the other components of the compositions.

The devices and kits may facilitate mixing of separated components prior to, during or immediately following discharge of the components from the device or kit.

In some embodiments, the compositions are bioinks and the device is a three-dimensional (3D) printer (e.g. an extrusion printer).

In some embodiments of the present invention, the crosslinking agent is riboflavin. In further embodiments, the riboflavin may be activated by UV light or blue light. The solution, which may contain type IV collagen and sodium and/or calcium ions, may be extruded in a line and UV light or blue light may be applied. Additional lines may be applied on top of the first line to form a structure which will be crosslinked by the crosslinking agent and the application of light.

In some embodiments, crosslinking occurs in under 15 minutes, under 14 minutes, under 13 minutes, under 12 minutes, under 11 minutes, under 10 minutes, under 9 minutes, under 8 minutes, under 7 minutes, under 6 minutes, under 5 minutes, under 4 minutes, under 3 minutes, under 2 minutes or under 1 minute. The light source used may be 3 mW/cm² 365 nm UV, 10 mW/cm² blue light or a tissue culture hood UV lamp.

The crosslinking agent may be Rose Bengal. In some embodiments, Rose Bengal is activated by green light. In further embodiments, Rose Bengal is activated by white light. In still further embodiments, crosslinking occurs in under 15 minutes, under 14 minutes, under 13 minutes, under 12 minutes, under 11 minutes, under 10 minutes, under 9 minutes, under 8 minutes, under 7 minutes, under 6 minutes, under 5 minutes, under 4 minutes, under 3 minutes, under 2 minutes or under 1 minute. The light source used may be 100 mw/cm² white light. The person skilled in the art would realise that the light source and parameters could be varied according to the particular application.

The collagen constructs of the present invention may have various thicknesses according to the application, for example, the thickness of a gel could be between 50 μm to 3 mm. The constructs may be injectable and/or mat be handled with tweezers without breaking.

### Applications of the Constructs

The present inventors have developed constructs which could be useful, for example, as replacement endothelial tissue. Non-limiting examples of tissue which the constructs could be used to repair/replace include the corneal endothelium and Descemet's membrane. The constructs could be used to treat corneal injury or disease by, for example, repairing/replacing human corneal endothelium and human Descemet's membrane. In some embodiments, the constructs could be used as a replacement for donor corneal tissue in endothelial keratoplasty or as an alternative to keratoplasty in endokeratoplasty. The constructs may also be used for the delivery of agent/s to target tissues and cells. Characteristics of the constructs make them highly suitable for bioprinting. The constructs of the present invention may be used in applications where there is a need for delivery of agents (e.g., natural growth factors, drugs, nanoparticles, and/or cells) and/or for the fixation of individual biological surfaces, and/or in tissue transplantation and/or in tissue culture methods.

In some embodiments the constructs can act as tissue sealants and/or as a fixative for biological structures. They may provide structural and/or nutritional support to tissue. Additionally or alternatively, the constructs may promote healing and/or facilitate the growth of target cell type/s, including those which may be provided as a component of the constructs and/or cells present in the target tissue. The compositions could be useful for culturing cells, for example, human corneal endothelial cells.

While no limitation exists as to the type of tissue to which the compositions may be applied, the present inventors have demonstrated that the compositions are compatible with cells, e.g., endothelial cells and/or may support the growth and/or proliferation of corneal endothelial cells.

For example, the compositions are demonstrated herein to be effective in supporting the growth and/or proliferation of corneal endothelial cells. In these embodiments, the compositions may be used to promote the proliferation and/or migration of corneal endothelial cells.

The present invention thus provides methods for the delivery of a variety of agents to biological targets. The targets may, for example, be located in or around eye tissue including tissue of the central and/or peripheral corneal epithelium and/or Descemet's membrane. The methods could also be used to repair and/or replace the collagen IV-containing basement layer of other human tissues, and/or to deliver agents to the collagen IV-containing basement layer of other human tissues.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### Examples

The present invention will now be described with reference to specific Examples, which should not be construed as in any way limiting.

### Example One: Generation of a Col-4 Membrane Mimicking Descemet's Membrane

Col-4 powder (minimum concentration of 6 mg/mL) was dissolved in 0.1M acetic acid, then neutralised with 5M NaOH and 27.4 mg/mL CaCl₂) to a final pH of between 6.7 and 7.4. These steps were conducted at RT. Once the solution was neutralised, 0.1-0.2 mg of riboflavin was added for every 90 μL of col-4 originally used and dissolved by further vortexing and centrifuging the col-4/riboflavin solution for 30 seconds and 1 minute, respectively.

With the addition of calcium ions, a clear and neutralised col-4 solution was generated. The resulting liquid could be photocrosslinked immediately or stored at −30° C. for use a minimum of 1 week later. The solution could be kept at RT for use a minimum of 1 hour later. Neutralised col-4 without riboflavin could be stored at 30° C. for use a minimum of 1 week later with no change in crosslinking properties.

Multiple forms of col-4 membrane were generated:

a: A double layer of col-4 (approximately 100 μm). This was generated by firstly pipetting 25 μL of col-4/riboflavin solution onto a glass coverslip (13 mm in diameter). The droplet was spread to cover the coverslip area and placed under UV or blue light for 1 minute. A further 25 μL of the solution was then pipetted onto the partially crosslinked solution and spread to cover the area again. The col-4 film was then crosslinked under UV or blue light for a further 3 minutes to create a double-layered crosslinked col-4 film. The layered nature of the film created is intended to increase its strength compared to a single layer of col-4 film.

b: A triple layer of col-4 (approximately 200 μm). This was generated in a similar method to the double layer of col-4. 25 μL of col-4/riboflavin solution was pipetted onto a glass coverslip and spread to cover the area. This layer was placed under UV or blue light for 1 minute. A further 25p L was pipetted onto the partially crosslinked solution and spread to cover the area again. The film with two layers was crosslinked for another minute before a final 25 μL was added and spread to fill the area again creating a triple-layered structure.

The col-4 membranes were then placed in 35 mm petri dishes containing 1×PBS and underwent 2×15 minute washes until they became transparent. These membranes could be stored in 1×PBS at 4° C. until use. In culturing experiments col-4 membranes were initially sterilised for 15 minutes under the UV light of a cell culture hood. Once sterilised the membranes were placed in the wells of a 12 well plate.

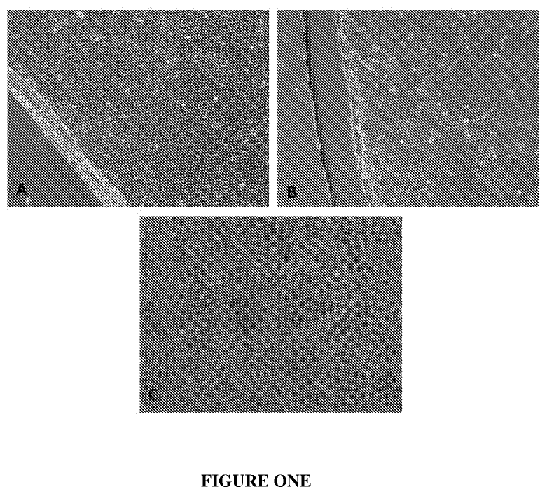

The double layered and triple layered col-4 membranes generated were both compatible with the corneal endothelial cells when tested using cell lines (FIG. 1A/B). In addition, increases in thickness seen between the single layer, double layer and triple layer col-4 membranes did not affect their ability to peel off from the well after 3-5 days of culture in 5% hPL media. No differences in transparency between the single layer and double/triple layer col-4 membrane was observed either macroscopically following washing or microscopically. Differences were observed when the double and triple layer col-4 membranes were handled with tweezers as the greater the number of layers crosslinked together the stronger the membrane structure was. However, while the triple layer col-4 was the strongest of those created, this strength compromised some of its flexibility as it could not be folded into a scroll. The double layer col-4 was stronger than the single layer and maintained an appropriate level of flexibility. Cells remained adhered to all types of membranes following mechanical disruption, an example of this is seen in FIG. 1C. The ideal bioengineered endothelium membrane appears to be a double layered membrane; but a triple layered membrane could also be a candidate.

### Development of a Unique Culturing System for Corneal Endothelial Cells on the Col-4 Membrane

Both an immortalised corneal endothelial cell line (B4G12) and primary corneal endothelial cells were used. B4G12 cells were thawed (after being stored in liquid nitrogen) and expanded in a coated tissue culture flask until they reached 80% confluence. Cells were cultured using a fetal calf serum (FCS) based growth medium of 5% FCS media in a 1:1 mixture of Nutrient Mixture Ham's F12 and Medium 199, 20 μg/mL ascorbic acid, 10 ng/mL bFGF & 10,000 U/mL penicillin & 10,000 μg/mL streptomycin. The cells were then passaged and counted. The cell density was adjusted so that 1×10⁵ cells was equivalent to between 75-100 μL of cell-laden medium. This amount of medium was added onto the generated col-4 membrane and the membrane with cells was left in the incubator for 45 minutes to allow for cell adhesion. A further 500 μL of FCS based growth medium was added to the well containing the membrane.

The following day, the FCS-based growth medium was replaced with 5% human platelet lysate (hPL) media. This hPL media had the same composition as the 5% FCS based growth medium except that FCS was replaced by hPL. The membrane was incubated in the hPL media for 3-5 days.

The cell line reached full confluence after 5 days with an average density of 3784 cells/mm². The generation of a corneal endothelial sheet during culturing of corneal endothelial cells on col-4 membranes required the addition of 5% hPL.

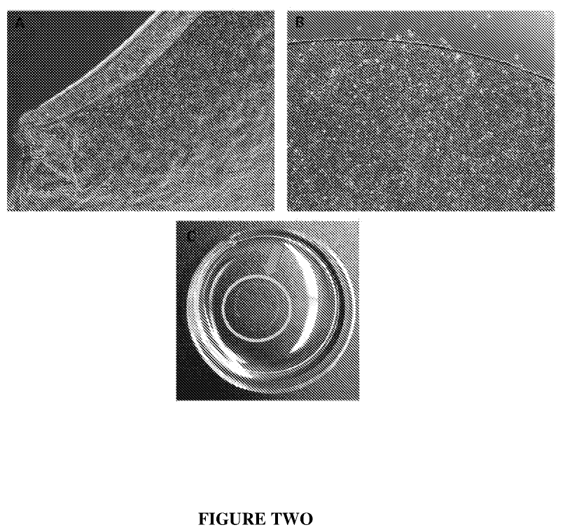

A total of 19 membranes; 3 triple layer and 16 double layer col-4 were created and observed. All of these membranes detached after the cells reached confluence in 5% hPL medium (FIG. 2A). All triple layer membranes created detached 4 days following the start of 5% hPL culture. The average detachment time for the double layer col-4 group, in which more membranes were created for observation, ranged from 3-5 days of culture in 5% hPL media (Table 1). In comparison, none of the col-4 corneal endothelial sheets cultured in 5% FCS media were able to detach (FIG. 2B).

TABLE 1
Proportions of double layer col-4 membranes
that detached 3-5 days following the start of culture

| | Percentage of double layer |
|---|---|
| Day of Detachment | col-4 sheets detached |
| Day 3 | 14/16 |
| Day 4 | 1/16 |
| Day 5 | 1/16 |

### Wound Healing Test

The bioengineered col-4 corneal endothelial sheets were subjected to mechanical disruption as surgeons would manipulate them using tweezers. Following mechanical trauma, including a scratch to the membrane, the sheets were pinned down to a 35 mm petri dish using tweezers and left to recover in 5% FCS media for 1 week. A comparison was made between recovery of cells over areas with col-4 remaining intact and areas with no col-4. Some sheets were torn to remove col-4 completely (control condition) while the cells were simply scratched off the col-4 in others. Each day the same areas of each sheet were imaged and the cell area recovered was determined using the image analysis software, Image J.

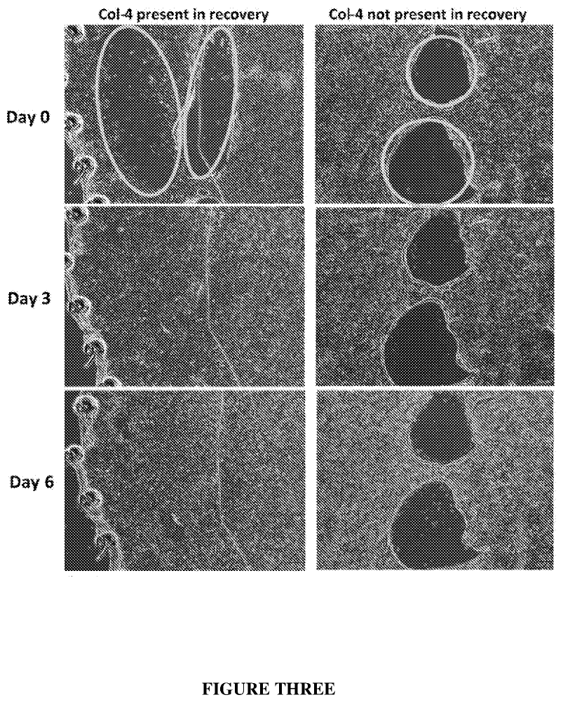

This test was important as commonly during DMEK surgeries 30% of cells are lost from the transplanted endothelium. After scratching, the endothelial cells were able to recover and formed a monolayer without scarring features on the col-4 membrane (FIG. 3) after 7 days. However, where there were tears in the col-4 layer itself the cells were unable to recover as there was no col-4 for them to expand onto.

### Mock Clinical Tests

### Marking the Bioengineered Endothelium

During surgery it is essential that the Descemet membrane can be marked to indicate the orientation so the surgeon can determine which side the endothelial cells are on so that they are correctly positioned for attachment to the patient's eye. A non-toxic pen was used to draw an asymmetrical shape such as an ‘F’ onto the col-4 membrane. The col-4 membrane with this mark was washed to determine whether the mark would remain visible following washes in various liquids and mechanical manipulation during surgery.

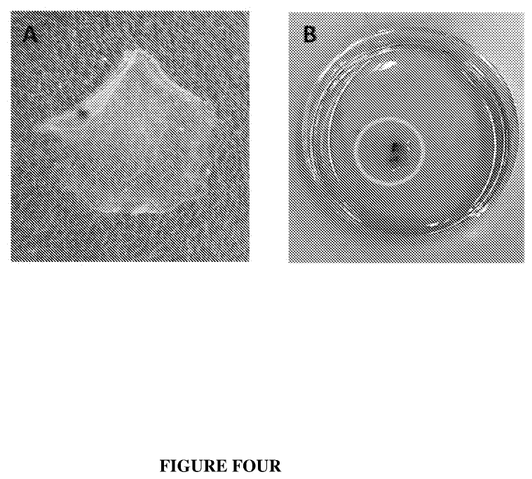

The col-4 membrane created could be marked with the same marker used in surgery to create asymmetrical marks that allow surgeons to distinguish which side the endothelial cells are on. Marks made on the non-endothelial side of the membrane were not able to be washed off and remained distinguishable (FIG. 4A). More complex shapes including the letter ‘F’ could also be drawn using dots to form a letter as directly drawing this shape onto the membrane could cause tears (FIG. 4B).

### Mock DMEK (Descemet's Membrane Endothelial Keratoplasty) Surgical Test

The surgical test was conducted in the surgical wet-lab using artificial eyes. The whole process followed the steps of normal DMEK surgery where a donor Descemet's membrane is transplanted with its attached endothelium. All three types of membranes with cells were used in this test i.e. single-layered, double-layered and triple-layered. The membranes were firstly trephined using an 8.5 mm trephine and stained with VisionBlue to allow for visualisation during the injection process. The membrane was then aspirated into a Stryker injector for insertion into an artificial eye. Following insertion, an air bubble was placed on top of the sheet in the eye to allow for unfurling and to mimic the part of DMEK surgery where the transplanted sheet is allowed to attach to the patient's eye.

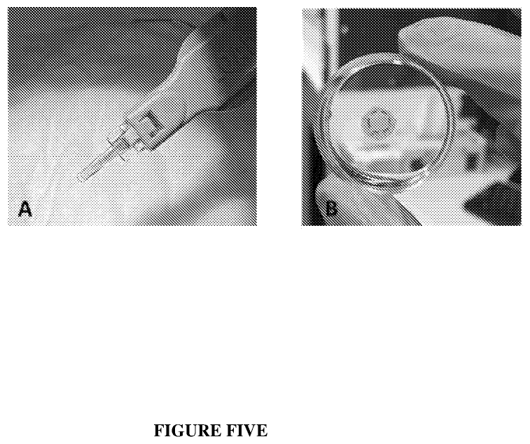

Both double layer and triple layer col-4 corneal endothelial sheets were injectable and ejectable into artificial eyes from Stryker injectors (shown in FIG. 5A). However, stronger forces were required for the triple layer membrane due to its increased strength and decreased flexibility. It could also be appropriately stained with VisionBlue dye (shown in FIG. 5B) which is used in normal surgeries for visualisation in the eye upon injection. Both col-4 corneal endothelial double and triple-layered sheets could be trephined appropriately to create the correct size sheet (this size shown in FIG. 5B by a dashed line). This trephining created a neat circular layer by removing uneven edges. They were also compatible with the big bubble technique and could be ejected properly into the artificial eyes and pig eyes.

### Ex Vivo Test

This test was conducted to determine adhesion of the col-4 corneal endothelial to a whole cornea lacking a Descemet's membrane. The sheet was placed into the eye and flattened with the cell side facing upward to mimic the normal structure and orientation of corneal layers. At 5 different time points; 1 min, 5 min, 10 min and 15 min the adhesion of the layer was determined by picking up the whole cornea and looking for movement of the layer from its original position.

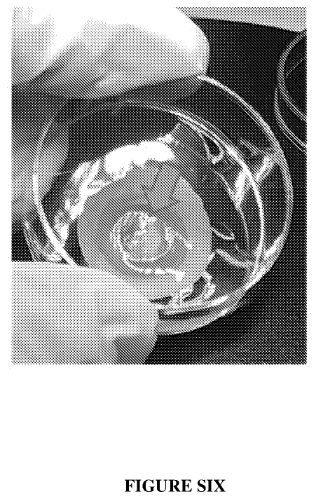

After the bioengineered endothelium layer was placed into a human cornea without a Descemet's membrane, it was able to adhere immediately to the stroma (within 1 minute) when there was minimal liquid in the eye (FIG. 6). This adhesion was maintained at all following time points checked. However, even after 1 hour of adhesion time with minimal liquid, upon the addition of a quantity of liquid the col-4 sheet dissociated from the cornea and floated to the surface. Following an overnight incubation in a humidified chamber the col-4 sheet was able to remain adhered to the corneal stroma upon addition of liquid. This adhesion was found to be permanent as following 2 weeks of culture in organ storage media the sheet remained attached. The sheet also remained transparent within the cornea both initially and over the period of its culture.

### Example Two: Preparation and Testing of Additional Endothelial Constructs

In this Example, additional endothelial constructs, including constructs using collagen I (col-1) and laminin as key component of the membranes, were prepared and tested. Primary endothelial cells were used to provide further evidence and examples of additional constructs.

### Materials and Methods

### Preparation of Collagen I (Col-1) Solution

Col-1 powder (Bovine skin, Sigma-Aldrich) was prepared as per the manufacturer's protocol. The powder was dissolved in 0.1 M acetic acid solution (pH=2.72) under continuous stirring at a speed of 800 rpm for 16 hours. The mixture was then observed by eye to confirm that the collagen powder was fully dissolved. The collagen solution was then transferred to a 1.5 ml Eppendorf tube and stored in a fridge.

90 parts of collagen solution was neutralised with 2.7 parts of 5 M NaOH and then mixed with 7.3 parts of CaCl₂). Riboflavin at 0.1% and 0.2% (w/v) solutions were made by adding 0.5 and 1 mg of riboflavin powder individually to 500 μL of 2 mg/ml CaCl₂ solution. The tubes were vortexed for 2 mins, and precipitation was checked by centrifuging the tubes in a mini centrifuge for 1 min. Riboflavin powder (Sigma-Aldrich) was added to the freshly made neutralised collagen solutions with ions added at a final concentration of 1 mg/ml. Col-1 solutions used in this Example had col-1 concentrations of between 3-12 mg/mL.

### Preparation of Collagen 4 (Col-4) Solution

Col-4 powder (minimum concentration of 6 mg/mL) was dissolved in 0.1M acetic acid, then neutralised with 5M NaOH and 27.4 mg/mL CaCl₂) to a final pH of between 6.7 and 7.4. These steps were conducted at RT. Once the solution was neutralised, 0.1-0.2 mg of riboflavin was added for every 90 μL of col-4 originally used and dissolved by further vortexing and centrifuging the col-4/riboflavin solution for 30 seconds and 1 minute, respectively.

With the addition of calcium ions, a clear and neutralised col-4 solution was generated. The resulting liquid could be photocrosslinked immediately or stored at −30° C. for use a minimum of 1 week later. The solution could be kept at RT for use a minimum of 1 hour later. Neutralised col-4 without riboflavin could be stored at 30° C. for use a minimum of 1 week later with no change in crosslinking properties.

Preparation of Collagen I and/or IV Constructs

Four collagen constructs were prepared:
1a: Col-4 at 10.8 mg/mL
1b: Col-1 and col-4 mixed with col-1 at 4 mg/mL and col-4 at 7 mg/mL
1c: Col-1 layer (10.5 mg/mL) on the bottom with col-4 layer (10.5 mg/mL) on the top
1d: Col-4 10 mg/mL and laminin 10 μg/mL

All constructs were prepared in two layers. Briefly, 20 μL collagen bioink (col-4 only, col-1 only or col-1 and col-4 mixed) was dropped onto a 22 mm diameter coverslip and spread to fill the surface using a p100 pipette. The hydrogels were crosslinked with 480 nm light for a minimum of 1 min from a distance of 3 cm. This was followed by applying a second layer of 20 μL collagen bioink on top of the first. The completed membranes were crosslinked for another minimum of 4 min. The thicknesses of the membranes were measured by placing the membranes vertically under a microscope. The membranes were washed in sterile 1×PBS three times for 5 min each on a shaker and left at 4° C. overnight until clear. The membranes were used immediately or stored in a fridge until use. Sterilisation was performed by taking the membrane out of PBS and placing it under UV within a Class II biosafety cabinet for 15 min.

### Transparency Test

Following crosslinking, all hydrogels were washed three times with 1×PBS for 10 minutes. The membranes were then placed flat in a 24-well plate, filling up at least 90% of the well. The optical density of each well between 400-1000 nm was measured using a platereader (Tecan Safire II) and the % transmittance was obtained by 1 minus the average OD.

Immunostaining of the Collagen IV with Laminin Membrane

The membrane was fixed with 4% paraformaldehyde for 15 min and rinsed with PBS. The sample was blocked by 5% BSA/PBS for 30 min, and incubated with Anti-Laminin beta 1 antibody (1 in 100 dilution; Abcam ab256380) at 4° C. for overnight. After incubation, the membrane was rinsed by PBS and incubated with donkey anti-rabbit 594 secondary antibody for 2 hours at room temperature (RT). The immunostaining was visualised and imaged using a Zeiss LSM700 scanning laser confocal microscope.

### Primary Corneal Endothelial Cell Culture

Primary corneal endothelial cells were cultured. Descemet's membrane was stripped from donor cornea and digested in collagenase A (2 mg/mL, Sigma) in a tube for 3-5.5 hours at 37° C. The digestion tube was centrifuged for 5 min at 190 g at room temperature (RT). Supernatants were removed and TrypLE select 1× (ThermoFisher) was added for further digestion for 5 min. M5 medium consists of 5% FBS, human endothelial serum-free media (SFM, ThermoFisher), 10 μM Y-27632 (Sigma) and 1% Antibiotic-Antimycotic (ThermoFisher) was added to inactivate TrypLE, and the tube was centrifuged again. Cell pellet was resuspended in fresh M5 media. Fifty μL of cell suspension (1.5×10\hat{ }5 cells) was either added to the centre of each membrane which was already placed in a 6 well, or a col-1 pre-coated petri dish with a 9 mm diameter spacer as the control. The plate and dish were placed into the incubator for 45 min, followed by the addition of 1.5 mL M5 media. The next day, M5 was replaced by proliferation medium M4 (Ham's F12:M199, 1:1, 5% FBS, 1% ITS, 10 μM Y-27632, 10 ng/ml hFGFb and 1% Antibiotic-Antimycotic). The medium was changed 3 times per week, and the cultures were examined by phase contract light microscopy.

### Immunostaining of Bioengineered Endothelium

Following culturing, the membranes and control were fixed with cold methanol for 2 minutes and rinsed with PBS. The samples were blocked by 5% BSA/PBS for 30 minutes and incubated with mouse Na/K-ATPase (Abcam) at 4° C. for overnight. The samples were rinse with PBS and incubated with Goat anti-mouse 488 secondary antibody and Hoechst 33342 for 2 hours at room temperature. The immunostaining was visualised and imaged using a Zeiss LSM700 scanning laser confocal microscope.

### Alternative Ways of Fabricating Collagen Constructs Using a Moulding Platform

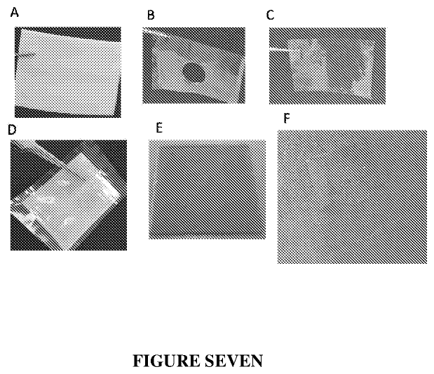

A moulding platform was assembled with three layers: the supporting layer made of either hydrophobic materials (e.g., silicone, parafilm) or a cold surface (e.g., cold metal); the intermediate layer which was a polyester film of 50 μm or more, with a hollowed centre; the top layer was another polyester film (FIG. 7).

Briefly, the supporting and intermediate layers were assembled first, and 30 μL collagen bioink (col-4 only, col-1 only or col-1 and col-4 mixed) was pipetted into the hollow centre of the intermediate layer. The top layer was then placed on top of the apparatus. The whole moulding was compressed with weight and the moulded hydrogel membrane was crosslinked with 480 nm light for 2 min from a distance of 3 cm.

After crosslinking, the top layer was removed, and 20 μL collagen bioink was applied to the crosslinked membrane. The bioink was then covered by the top layer and crosslinked again. The two-layered membrane was then removed from the mounding device and the thickness of membrane were measured by placing membrane vertically under a microscope.

The membrane was washed in sterile 1×PBS three times for 5 min each on a shaker and left at 4° C. overnight until clear. The membranes were used immediately or stored in a fridge until use. Sterilisation was performed by taking the membrane out of PBS and placing it under UV within a Class II biosafety cabinet for 15 min.

### Results

Preparation of Collagen I and/or IV Constructs

All four membranes were successfully constructed with an average thickness of 250 μm and transparency over 90% (Table 2).

TABLE 1
Summary of the characteristics of the collagen membranes constructed

| | Concentration | | % |
|---|---|---|---|
| Collagen type | (mg/ml) | Thickness | Transmittance |
| Col_IV | 10.8 | 214 | 92.6% |
| Col_IV and Col_I | 5.4 (IV and I) | 251 | 95.6% |
| (mixture) | | | |
| Col_IV | 8 | 277 | 92.80% |
| Collagen I layer with | 10.8 (IV) 10.8 (I) | 224 | 91% |
| collagen IV layer | | | |
| Col_IV and Laminin | 10.8 (IV) 0.01 | 196 | 92.20% |
| | (laminin) | | |

Immunostaining of the Collagen IV with Laminin Membrane

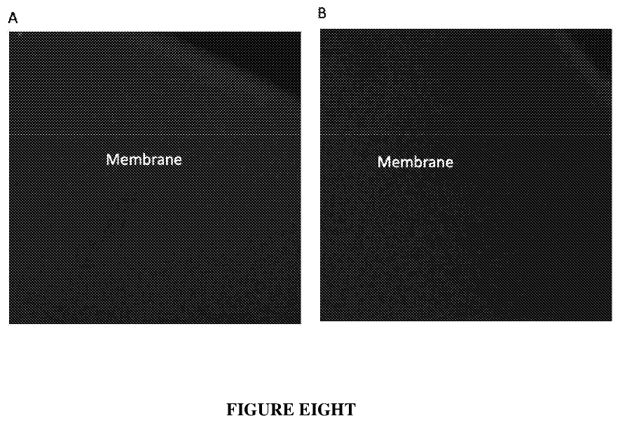

Fluorescent images of the collagen membrane with laminin showed stronger staining than the control (FIG. 8). This suggests that laminin (red fluorescence) was incorporated across the membrane.

### Primary Corneal Endothelial Cell Culture

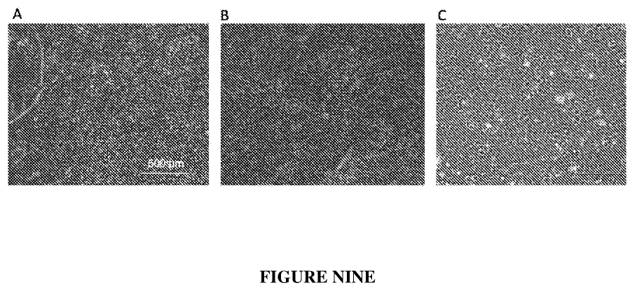

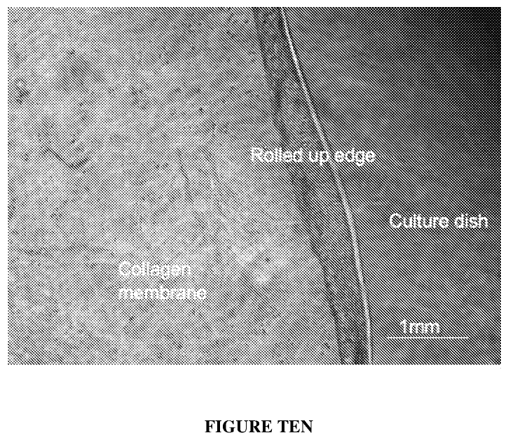

Primary corneal endothelial cells reached confluence for all membranes tested (FIG. 9). At about 10-14 days of culturing, the membranes could self-detach from the coverslip they were prepared on (FIG. 10). The detached membranes were then picked up with tweezers and transferred to another dish for immunostaining.

### Immunostaining of Bioengineered Endothelium

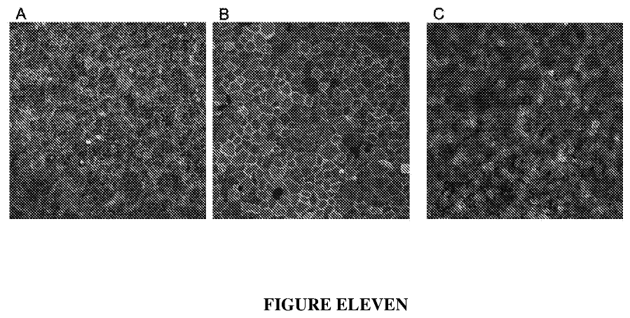

Primary human corneal endothelial cells grown on col-1 only and col-1 I and col-4 layered membranes expressed higher intensities of the key marker Na/K-ATPase on the cell border compared to the control (col-1 pre-coated petri dish) (FIG. 11).

### Alternative Ways of Fabricating Collagen Constructs Using a Moulding Platform

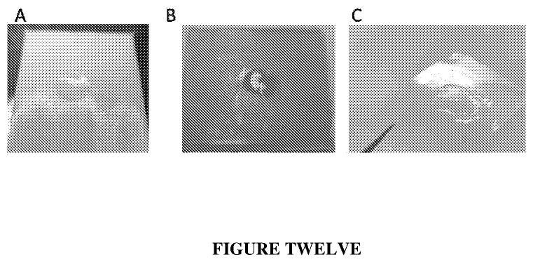

Two layered collagen membranes could be made successfully by the moulding platform (FIG. 12). When a cold metal plate was used, the membrane adhered to the top layer rather than the supporting material. However, the adhesion was weak and the membrane could be easily removed by rinsing with PBS. The moulding platform method allowed for consistent membrane generation. The top layer ensured the smooth surface of the membrane, and the membrane could be removed from the supporting material easily.

### CONCLUSION

The data presented in this Example show that a col-4 membrane can incorporate col-1 and structural proteins such as laminin without affecting the transparency of the membrane. In addition, a two-layered construct can be made using one layer of pure col-1 and another layer of col-4 through the crosslinking method. Primary corneal endothelial cells were tested and the membranes could self-detach from the disc that they were produced on after cells reached confluence (usually at about 10 days of culturing). There is no need to add human platelet lysate when culturing primary endothelial cells. New ways of fabricating collagen constructs were also developed that generated smooth surfaces on the membranes and allowed easy removal and handling of the membranes.

1. A construct comprising a composition, wherein the composition comprises:
type IV collagen;
one or more crosslinking agents;
endothelial cells; and
platelet lysate and/or a component thereof.

2. A construct comprising a composition, wherein the composition comprises:
type IV collagen;
one or more crosslinking agents; and
endothelial cells.

3. The construct of claim 1 or claim 2, wherein the endothelial cells are corneal endothelial cells.

4. The construct of any one of claims 1 to 3, wherein the endothelial cells are human endothelial cells.

5. The construct of any one of claims 1, 3 or 4, wherein the platelet lysate is human platelet lysate.

6. The construct of any one of claims 1 or 3 to 5, wherein the component of platelet lysate is any one or more of: fibrinogen, basic fibroblast growth factor (bFGF), transforming growth factor (TGF-β), insulin-like growth factor (IGF-1), brain-derived neurotrophic factor (BDNF), vascular endothelial growth factor (VEGF), epidermal growth factor (EGF), hepatocyte growth factor (HGF), platelet-derived growth factor (PDGF), IgG, albumin.

7. The construct of any one of claims 1 or 3 to 6, wherein the component of platelet lysate is fibrinogen.

8. The construct of claim 7, wherein the fibrinogen is human fibrinogen.

9. The construct of any one of claims 1 to 8, wherein the construct and/or the composition further comprises sodium ions and/or calcium ions.

10. The construct of any one of claims 1 to 9, wherein the one or more crosslinking agents are capable of activation by UV light, blue light, green light or white light.

11. The construct of any one of claims 1 to 10, wherein the one or more crosslinking agents comprise riboflavin.

12. The construct of any one of claims 1 to 11, wherein the construct comprises at least one layer comprising the type IV collagen and the one or more crosslinking agents, and wherein each layer is produced by crosslinking a solution comprising:
6-24 mg/ml type IV collagen, and
0.04-0.15 M sodium ions and/or 0.008-0.4 M calcium ions.

13. The construct of claim 12, wherein the solution comprises:
(i) 6-24 mg/ml type IV collagen, 0.06-0.1 M sodium ions, and 0.01-0.04 M calcium ions; or
(ii) 3-15 mg/ml type IV collagen, 0.06-0.08 M sodium ions, and 0.015-0.03 M calcium ions; or
(iii) 4-12 mg/ml type IV collagen, 0.06-0.07 M sodium ions, and 0.018-0.02 M calcium ions.

14. The construct of claim 12 or claim 13, wherein the solution comprises:
(i) less than 24 mg/ml type IV collagen;
(ii) more than 0.04 M sodium ions; and
(iii) more than 0.008 M calcium ions.

15. The construct of any one of claims 12 to 14, wherein the solution comprises 0.01-0.1 mg riboflavin.

16. The construct of any one of claims 1 to 15, wherein the construct comprises two or more layers comprising the type IV collagen and the one or more crosslinking agents, and wherein each of the two or more layers has been crosslinked to at least one other layer.

17. The construct of any one of claims 12 to 16, wherein the construct comprises at least one additional layer comprising type I collagen and one or more crosslinking agents, wherein the additional layer is produced by crosslinking a solution comprising:
3-15 mg/ml type I collagen;
0.135-0.5 M sodium ions and/or 0.008-0.4 M calcium ions.

18. The construct of claim 17, wherein each layer has been crosslinked to at least one other layer.

19. The construct of any one of claims 1 to 18, wherein the construct comprises a first layer comprising the collagen and the one or more crosslinking agents that has been individually crosslinked prior to crosslinking to one or more other layers.

20. The construct of any one of claims 1 to 11, wherein the construct comprises at least one layer comprising type IV collagen, type I collagen and the one or more crosslinking agents, and wherein each layer is produced by crosslinking a solution comprising:
6-24 mg/ml type IV collagen, and
3-15 mg/ml type I collagen.

21. The construct of claim 20, wherein the solution comprises 0.01-0.1 mg riboflavin.

22. The construct of claim 20 or claim 21, wherein the construct comprises two or more layers comprising the type IV collagen, type I collagen and the one or more crosslinking agents, and wherein each of the two or more layers has been crosslinked to at least one other layer.

23. The construct of any one of claims 20 to 22, wherein the construct comprises a first layer comprising the collagen and the one or more crosslinking agents that has been individually crosslinked prior to crosslinking to one or more other layers.

24. The construct of any one of claims 1 to 23, wherein the construct and/or the composition further comprises mammalian cells.

25. The construct of claim 24, wherein the mammalian cells comprise or consist of human cells.

26. The construct of any one of claims 1 to 25, wherein the construct and/or the composition further comprises any one or more of: a culture medium, growth factors, hormones, matrix proteins, glycoproteins, vitamins, ions, ion sources, fibronectin, amino acids, antibiotics, anaesthetics, factor XIII, Fetal Bovine Serum (FBS), Fetal Calf Serum (FCS), human serum, platelet lysate, human platelet lysate, therapeutic drugs.

27. The construct of claim 26, wherein the construct and/or the composition comprises a culture medium comprising the ions and amino acids.

28. The construct of claim 26 or claim 27, wherein:
(i) the growth factors comprise VEGF and/or FGF; and/or
(ii) the vitamins comprise riboflavin; and/or
(iii) the matrix proteins comprise type I collagen; and/or
(iv) the matrix proteins comprise laminin.

29. The construct of any one of claims 1 to 28, wherein the construct and/or the composition comprises ions, and wherein the ions are components of an ionic salt included in the construct and/or the composition.

30. The construct of any one of claims 1 to 29, wherein the type IV collagen is neutralised.

31. The construct of any one of claims 1 to 30, wherein the construct and/or the composition further comprises any one or more of: type VIII collagen, laminin, nidogen, perlecan.

32. A method of preparing a construct, the method comprising:
(i) providing a composition, wherein the composition is produced by crosslinking a solution comprising:
type IV collagen, and
one or more crosslinking agents; and
(ii) adding:
endothelial cells, and
platelet lysate and/or a component thereof.

33. A method of preparing a construct, the method comprising:
(i) providing a composition, wherein the composition is produced by crosslinking a solution comprising:
type IV collagen, and
one or more crosslinking agents; and
(ii) adding endothelial cells.

34. A method of culturing endothelial cells, the method comprising:
(i) providing a composition, wherein the composition is produced by crosslinking a solution comprising:
type IV collagen, and
one or more crosslinking agents; and
(ii) adding:
endothelial cells, and
platelet lysate and/or a component thereof.

35. A method of culturing endothelial cells, the method comprising:
(i) providing a composition, wherein the composition is produced by crosslinking a solution comprising:
type IV collagen, and
one or more crosslinking agents; and
(ii) adding endothelial cells.

36. The method of any one of claims 32 to 35, wherein the composition is produced by crosslinking a second solution comprising:
type I collagen, and
one or more crosslinking agents,
prior to (ii).

37. The method of any one of claims 32 to 35, wherein the solution further comprises type I collagen.

38. The method of any one of claims 32 to 37, wherein the endothelial cells are corneal endothelial cells.

39. The method of any one of claims 32, 34 or 36 to 38, wherein the platelet lysate is human platelet lysate.

40. The method of any one of claims 32, 34 or 36 to 39, wherein the component of platelet lysate is any one or more of: fibrinogen, bFGF, TGF-β, IGF-1, BDNF, VEGF, EGF, HGF, PDGF, IgG, albumin.

41. The method of any one of claims 32, 34 or 36 to 40, wherein the component of platelet lysate is fibrinogen.

42. The method of claim 41, wherein the fibrinogen is human fibrinogen.

43. The method of any one of claims 32 to 42, further comprising adding sodium ions and/or calcium ions in step (i) and/or step (ii).

44. The method of any one of claims 32 to 43, wherein the one or more crosslinking agents are capable of activation by UV light, blue light, green light or white light.

45. The method of any one of claims 32 to 44, wherein the one or more crosslinking agents comprise riboflavin.

46. The method of any one of claims 32 to 45, wherein the composition comprises at least one layer comprising the crosslinked solution, and wherein the solution comprises:
6-24 mg/ml type IV collagen, and
0.04-0.15 M sodium ions and/or 0.008-0.4 M calcium ions.

47. The method of claim 46, wherein the solution comprises:
(i) 6-24 mg/ml type IV collagen, 0.06-0.1 M sodium ions, and 0.01-0.04 M calcium ions; or
(ii) 3-15 mg/ml type IV collagen, 0.06-0.08 M sodium ions, and 0.015-0.03 M calcium ions; or
(iii) 4-12 mg/ml type IV collagen, 0.06-0.07 M sodium ions, and 0.018-0.02 M calcium ions.

48. The method of claim 46 or claim 47, wherein the solution comprises:
(i) less than 24 mg/ml type IV collagen;
(ii) more than 0.04 M sodium ions; and
(iii) more than 0.008 M calcium ions.

49. The method of any one of claims 45 to 48, wherein the solution comprises 0.01-0.1 mg riboflavin.

50. The method of any one of claims 32 to 49, wherein the composition comprises two or more layers comprising the collagen and the one or more crosslinking agents, and wherein each of the two or more layers has been crosslinked to at least one other layer.

51. The method of any one of claims 32 to 50, wherein the composition comprises a first layer comprising the collagen and the one or more crosslinking agents that has been individually crosslinked prior to crosslinking to one or more other layers.

52. The method of claim 50 or claim 51, wherein at least one layer of the composition is crosslinked on a hydrophobic material.

53. The method of claim 52, wherein the hydrophobic material is silicone or parafilm.

54. The method of claim 50 or claim 51, wherein at least one layer of the composition is crosslinked on a cold material.

55. The method of claim 54, wherein the cold material is cold metal.

56. The method of any one of claims 32 to 55, further comprising adding mammalian cells in step (i) and/or step (ii).

57. The method of claim 56, wherein the mammalian cells comprise or consist of human cells.

58. The method of any one of claims 32 to 57, further comprising adding in step (i) and/or step (ii) any one or more of: a culture medium, growth factors, hormones, matrix proteins, glycoproteins, vitamins, ions, ion sources, fibronectin, amino acids, antibiotics, anaesthetics, factor XIII, FBS, FCS, human serum, platelet lysate, human platelet lysate, therapeutic drugs.

59. The method of claim 58, wherein the ions and/or amino acids are provided in a culture medium.

60. The method of claim 58 or claim 59, wherein:
(i) the growth factors comprise VEGF and/or FGF; and/or
(ii) the vitamins comprise riboflavin; and/or
(iii) the matrix proteins comprise type I collagen; and/or
(iv) the matrix proteins comprise laminin.

61. The method of any one of claims 32 to 60, further comprising adding ions as components of an ionic salt in step (i) and/or step (ii).

62. The method of any one of claims 32 to 61, wherein the type IV collagen is neutralised.

63. The method of any one of claims 32 to 62, further comprising adding in step (i) and/or step (ii) any one or more of: type VIII collagen, laminin, nidogen, perlecan.

64. A construct obtained or obtainable by the method of any one of claims 32, 33 or 36 to 63.

65. A method of replacing endothelial tissue, the method comprising applying the construct of any one of claims 1 to 31 or claim 64.

66. The method of claim 65, wherein the endothelial tissue comprises or consists of the corneal endothelium.

67. The method of claim 65 or claim 66, wherein the endothelial tissue is human endothelial tissue.

68. The method of any one of claims 65 to 67, wherein the endothelial tissue comprises or consists of Descemet's membrane.

69. A method of treating corneal injury or disease, the method comprising applying the construct of any one of claims 1 to 31 or claim 64.

70. A method of delivering agents to tissue, the method comprising applying the composition of any one of claims 1 to 31 or claim 64 to the tissue.

71. A construct of any one of claims 1 to 31 or claim 64 for use in replacing endothelial tissue.

72. The use of claim 71, wherein the endothelial tissue comprises or consists of the corneal endothelium.

73. The use of claim 71 or claim 72, wherein the endothelial tissue is human endothelial tissue.

74. The use of any one of claims 71 to 73, wherein the tissue comprises or consists of Descemet's membrane.

75. A construct of any one of claims 1 to 31 or claim 64 for use in treating corneal injury or disease.

76. A construct of any one of claims 1 to 31 or claim 64 for use in delivering agents to tissue.

77. A kit, package or device for producing a construct comprising a composition, wherein the composition comprises:
type IV collagen, and
one or more crosslinking agents;
and wherein the kit, package or device further comprises:
endothelial cells, and
platelet lysate and/or a component thereof.

78. A kit, package or device for producing a construct comprising a composition, wherein the composition comprises:
type IV collagen, and
one or more crosslinking agents;
and wherein the kit, package or device further comprises endothelial cells.

79. The kit, package or device of claim 77 or claim 78, wherein the composition further comprises type I collagen.

80. The kit, package or device of claim 77 or claim 78, wherein the kit, package or device is for producing a further composition, wherein the composition comprises:
type IV collagen,
type I collagen, and
one or more crosslinking agents.

81. Use of a kit, package or device for preparing a construct comprising a composition, wherein the composition comprises:
type IV collagen, and
one or more crosslinking agents;
and wherein the kit package or device further comprises:
endothelial cells, and
platelet lysate and/or a component thereof.

82. Use of a kit, package or device for preparing a construct comprising a composition, wherein the composition comprises:
type IV collagen, and
one or more crosslinking agents;
and wherein the kit package or device further comprises:
endothelial cells.

83. The use of claim 81 or claim 82, wherein the composition further comprises type I collagen.

84. The use of claim 81 or claim 82, wherein the use is for producing a further composition, wherein the composition comprises:
type IV collagen,
type I collagen, and
one or more crosslinking agents.

85. The kit, package or device of any one of claims 77 to 80 or the use of any one of claims 80 to 84, wherein the endothelial cells are corneal endothelial cells.

86. The kit, package or device of any one of claims 77 to 80 or 85 or the use of any one of claims 80 to 85, wherein the endothelial cells are human endothelial cells.

87. The kit, package or device of any one of claims 77, 79, 80, 85 or 86 or the use of any one of claims 80, 81 or 83 to 86, wherein the platelet lysate is human platelet lysate.

88. The kit, package or device of any one of claims 77, 79, 80 or 85 to 87 or the use of any one of claims 80, 81 or 83 to 87, wherein the component of platelet lysate is any one or more of: fibrinogen, bFGF, TGF-β, IGF-1, BDNF, VEGF, EGF, HGF, PDGF, IgG, albumin.

89. The kit, package or device of any one of claims 77, 79, 80 or 85 to 88 or the use of any one of claims 80, 81 or 83 to 88, wherein the component of platelet lysate is fibrinogen.

90. The kit, package or device or the use of claim 89, wherein the fibrinogen is human fibrinogen.

91. The kit, package or device of any one of claims 77 to 80 or 85 to 90 or the use of any one of claims 80 to 90, wherein the construct and/or the composition further comprises sodium ions and/or calcium ions.

92. The kit, package or device of any one of claims 77 to 80 or 85 to 91 or the use of any one of claims 80 to 91, wherein the one or more crosslinking agents are capable of activation by UV light, blue light, green light or white light.

93. The kit, package or device of any one of claims 77 to 80 or 85 to 92 or the use of any one of claims 80 to 92, wherein the one or more crosslinking agents comprise riboflavin.

94. The kit, package or device of any one of claims 77 to 80 or 85 to 93 or the use of any one of claims 80 to 93, wherein the construct comprises at least one layer comprising the type IV collagen and the one or more crosslinking agents, and wherein each layer is produced by crosslinking a solution comprising:
6-24 mg/ml type IV collagen, and
0.04-0.15 M sodium ions and/or 0.008-0.4 M calcium ions.

95. The kit, package or device or the use of claim 94, wherein the solution comprises:
(i) 6-24 mg/ml type IV collagen, 0.06-0.1 M sodium ions, and 0.01-0.04 M calcium ions; or
(ii) 3-15 mg/ml type IV collagen, 0.06-0.08 M sodium ions, and 0.015-0.03 M calcium ions; or
(iii) 4-12 mg/ml type IV collagen, 0.06-0.07 M sodium ions, and 0.018-0.02 M calcium ions.

96. The kit, package or device or the use of claim 94 or claim 95, wherein the solution comprises:
(i) less than 24 mg/ml type IV collagen;
(ii) more than 0.04 M sodium ions; and
(iii) more than 0.008 M calcium ions.

97. The kit, package or device or the use of any one of claims 94 to 96, wherein the solution comprises 0.01-0.1 mg riboflavin.

98. The kit, package or device of any one of claims 77 to 80 or 85 to 97 or the use of any one of claims 80 to 97, wherein the construct comprises two or more layers comprising the type IV collagen and the one or more crosslinking agents, and wherein each of the two or more layers has been crosslinked to at least one other layer.

99. The kit, package or device of any one of claims 77 to 80 or 85 to 98 or the use of any one of claims 80 to 98, wherein the construct comprises a first layer comprising the type IV collagen and the one or more crosslinking agents that has been individually crosslinked prior to crosslinking to one or more other layers.

100. The kit, package or device of any one of claims 77 to 80 or 85 to 99 or the use of any one of claims 80 to 99, wherein the construct and/or the composition further comprises mammalian cells.

101. The kit, package or device or the use of claim 100, wherein the mammalian cells comprise or consist of human cells.

102. The kit, package or device of any one of claims 77 to 80 or 85 to 101 or the use of any one of claims 80 to 101, wherein the construct and/or the composition further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, FBS, FCS, human serum, platelet lysate, human platelet lysate, therapeutic drugs.
